(19) United States
(12) Patent Application Publication
(10) Pub. No.: US 2022/0218466 A1
(43) Pub. Date: Jul. 14, 2022

(54) Accommodating Intraocular Lenses with Combination of Mechanical Driving Components

(71) Applicant: AKKOLENS INTERNATIONAL B.V., Breda (NL)

(72) Inventors: Michiel Christiaan Rombach, Breda (NL); Willem Pieter Van Lawick, Breda (NL)

(21) Appl. No.: 17/610,913

(22) PCT Filed: May 14, 2020
(86) PCT No.: PCT/NL2020/050307

§ 371 Date: Nov. 12, 2021

(30) Foreign Application Priority Data
May 15, 2019 (NL) 2023139

(51) Int. Cl. A61F 2/16

(52) U.S. Cl. CPC A61F 2/1629; A61F 2002/1682; A61F 2/1632

(57) ABSTRACT

Accommodating intraocular lens with an variable power lens with a combination of mechanical driving components which can include a barrel driving component to transfer lateral movement from a driving component in the eye to the variable power lens, and/or a, novel, flange driving component and/or a, novel, bouncing chamber driving component to, firstly, translate axial movement of the ciliary mass and/or the zonular system into lateral movement and, secondly, transfer this lateral movement to the variable power lens.

### CROSS-REFERENCE TO RELATED APPLICATIONS

This application is the United States national phase of International Application No. PCT/NL2020/050307 filed May 14, 2020, and claims priority to Netherlands Patent Application No. 2023139 filed May 15, 2019, the disclosures of which are hereby incorporated by reference in their entirety.

### BACKGROUND OF THE INVENTION

### 1. Field of the Invention

Accommodating intraocular lenses restore the accommodation of the human eye, meaning, these lenses provide the retina with a sharp image of an object at different distances, from a far distance, say, infinity, to near distance, say, reading distance, by adjusting the optical power of the accommodating intraocular lens.

### 2. Discussion of the Related Art

Accommodating intraocular lenses, also: accommodating lenses, as presented in this present document include, for optical functioning, at least one variable power lens which comprises at least two optical surfaces which lens component optically modifies/adjusts at least one incoming light beam, generally, but not restricted to, a modification of defocus power. The lens can be a variable power lens component, also: variable power lens, providing variable optical power which component can comprise a single lens optical element, also: lens element, for example a radially flexible lens component, also: flexible lens component, or, alternatively, which variable power lens comprises multiple lens optical elements, for example two laterally or axially moving optical elements. Alternatively, the lens can be a fixed power lens component which component comprises a single optical element. The accommodating intraocular lens also comprises, for mechanical functioning, at least one mechanical construction which construction comprises at least one mechanical component which transfers movement of at least one eye component, an anatomical component of the eye to the variable power lens, or, alternatively, the mechanical component, firstly, translates the direction of movement of the eye component into any other direction, as in the present invention, in which the mechanical component translates axial movement, a movement in a direction largely along the optical axis, of the eye component into a lateral movement, a movement in a direction largely perpendicular to the optical axis and, secondly, which mechanical component, comprising at least one driving means, transfers the translated movement to the variable power lens.

Furthermore, the accommodating lens can also be coupled to at least one intraocular artificial mechanical driving component, for example, a MEMS, a micro-electro-mechanical system, and/or, the variable power lens can comprise any intraocular artificial variable power lens, for example, an optical system including at least one artificial liquid crystal lens with such artificial components and constructions driven, for example, providing leverage of movements of the movements of the eye component, and/or, controlled by movement of at least one eye component via at least one mechanical component as disclosed in the present document.

The documents, references, cited in this document are considered part of this document as well as citations to related documents therein.

Accommodating lenses can vary the optical power by, for example, axial movement, meaning: movement in the direction along the optical axis, of at least one lens component, for example, movement of fixed focus intraocular lens components, as disclosed in, for example, US2019053893 and WO2006NL50050 (EP1871299), or, alternatively, axial movement of multiple, positive largely spherical, lens components in opposite directions, as disclosed in, for example, US2018221139 and US2013013060 (CA2849167, US2002138140).

Movements, including axial movements, of lens components can be driven by the ciliary muscle, generally via the remains, the rim, of the capsular bag, as in US2019053893, or, alternatively, such movement can be driven by the iris, as in, for example, WO2019027845, ES2650563 and US2008215146, or, alternatively, such movement can be driven by the ciliary mass directly, as in, for example, US2018353288. Note that the term ‘ciliary mass’ as used in the present document can, but not exclusively so, include the ‘zonular system’ which zonulae connect the the ciliary mass to the capsular bag.

Alternatively, a gradually progressive lens component, for example, a lens with a single cubic free-form surface or, alternatively, a stepwise progressive lens component lens, for example, a bifocal or trifocal lens component can be moved in a lateral direction, as in US201010624. Radially flexible lens components, elastic lenses which can change radial thickness, can provide a variable focus when compressed laterally, as in, for example, AU2014236688, US201562257087 and US2018256315, AU2014236688, discloses lens components in which an elastic container contains a fluid, for example, an oil, or, alternatively, as in US2018344453, DE11200900492, U.S. Ser. No. 10/004,595, US2018271645, US2019015198 and U.S. Pat. No. 9,744,028 which disclose a change in shape of a uniform elastic lens component and, alternatively, as in US2019000162 which discloses, in this particular case, a flexible lens component driven by pressure of the posterior vitreous of the eye. US2012310341, US2011153015 and DE112009001492 disclose any type of shape changing lenses, radially flexible lenses, which lenses are positioned at the sulcus plane instead of inside the remains of the capsular bag of the eye with such change of shape driven directly by the ciliary mass or zonulae system of the eye, or, alternatively, by the iris, or, alternatively, by the sclera, for example by the sulcus root which is directly connected to the sclera of the eye.

Note that lateral movement a lens can be a parallel mutual shift of lens components which is as used as the main example of variable power lens in this document, but also a rotation of at least one component as in the rotation of lens components comprising at least two chiral optical surfaces in a lateral plane, WO2014058315 and ES2667277, or, alternatively, a combination of wedging and rotation of at least two complex free-form surfaces, for example adapted cubic optical surfaces, as in, for example, US2012323321 in a lateral plane.

Accommodating lenses can comprise mechanical components translate lateral compression, compression perpendicular to the optical axis, of the mechanical construction into mutual movement of the lens components and thus provide variable optical power as disclosed in, for example, US2010106245 and multiple other documents cited above. Such construction can comprise at least one flexible lens component of which the power depends on the degree of change of shape, radial flex, of the elastic lens component as disclosed in, for example, but not limited hereto, US2011153015 and US2019015198. Such construction must comprise a mechanical component to provide translation of lateral movement of the construction into radial flex of the radially flexible lens component. component.

Accommodating lenses are can be implanted at the sulcus plane and ciliary plane of the eye, both positions meaning in front of, anterior of, the capsular bag of the eye and comprise at least one mechanical component providing translation of movement of the ciliary mass or zonulae or any other related anatomical structure of the eye into mutual translation of, elastically stiff, lens components or, alternatively, in a change of shape of, elastically flexible, material.

The lens component can also comprise at least one additional optical surface to provide correction of at least one optical aberration, an aberration other than defocus, of the eye. For example, fixed optical power can correct a fixed power aberration, for example residual refractive error of the eye such as myopia, hyperopia or astigmatism of the eye, or any combination of such fixed power aberrations.

The lens construction can also comprise at least one additional optical surface to provide variable optical power to correct at least one, undesired, variable optical aberration of the eye other than the, desired, variable defocus. Such undesired variable aberration can be, for example, but not restricted hereto, variable aspherical aberration, or, variable astigmatism, or, variable coma, or, variable trefoil aberrations or any combination of any variable aberrations.

Such accommodating lenses are known from, for example, EP1720489, NL2015644, NL2012133, NL2012420 and NL2009596 and many related documents thereto. The lens construction should also comprise mechanical components, haptics, adapted to provide translation of lateral compression of the construction into mutual translation of the optical elements. The second lens construction can also comprise at least one additional optical surface to provide corrective optical power to correct at least one optical aberration of the eye, for example, provide fixed optical power to correct at least one fixed optical aberration of the eye, which can be a residual refractive error of the eye, or, alternative, which can be myopia, hyperopia or astigmatism of the eye. Also, the additional optical surface provide variable optical power to correct at least one variable optical aberration of the eye other than variable defocus, for example, undesired variable aspherical aberration, or add the same, in case this is desired. The residual refractive error of the eye can be myopia of the eye, or, hyperopia of the eye, or, astigmatism of the eye, with additional optical surface providing variable optical power to correct at least one variable optical aberration of the eye other than variable defocus, for example, the variable optical aberration of the eye is variable aspherical aberration.

Accommodating lenses are, preferably, implanted at the sulcus plane, or, alternatively, in the sulcus of the eye, and driven directly by the ciliary mass/zonula system so that posterior capsular opafication, PCO, and/or shrinkage of the capsular bag will not affect the accommodating properties of the accommodating lens. The variable power lens can comprise at least one flexible lens component adapted to provide variable optical power which power depends on the degree of change of shape of the flexible optical component. Such components are known from AU2014236688, U.S. Pat. No. 1,011,745 and US2018256311, which documents disclose a lens shaped elastic container filled with a fluid or a uniform flexible lens implanted in the rim of the capsular bag. US2019000612 discloses such lenses to be implanted at the sulcus plane, in front of the capsular bag.

### SUMMARY OF THE INVENTION

The invention disclosed in the present document concerns an accommodating intraocular lens with at least one variable power lens to provide variable optical power and at least one mechanical construction, also: haptic, which drives the variable power lens which mechanical construction can comprise a combination of at driving components including at least one driving component which transfers movement of the ciliary mass in a direction largely perpendicular to the optical axis, lateral movement, to the optical element, and/or, at least one driving component which translates movement of the ciliary mass in a direction largely along the optical axis, axial movement, into a lateral movement and transfers this lateral movement to the variable power lens.

The eye and accommodating lens have the same optical axis. The mechanical construction can comprise a combination of at least two driving components including at least one barrel driving component to transfer movement of the ciliary mass in a lateral direction to movement of the mechanical component in a lateral direction, and, at least one flange driving component to provide translation of movement of the ciliary mass in an axial direction into movement in a lateral direction and to provide transfer of the translated movement to movement of the mechanical component in a lateral direction.

The flange driving component can be a wedge shaped flange driving component, the preferred embodiment, which component translates movement of the ciliary mass in an axial direction into movement in a lateral direction by sliding in a lateral direction, in between the ciliary mass and the iris, with the sliding forced by closure of the sulcus due to forward axial movement of the ciliary mass. Alternatively, the flange driving component can be a bouncing chamber driving component which translates movement of the ciliary mass in an axial direction into movement in a lateral direction by elongation of the chamber in a lateral direction, with the chamber in between the ciliary mass and the iris and elongation forced by compression due to closure of the sulcus by the ciliary mass. Alternatively, the mechanical construction can comprise any combination of said wedge shaped flange driving component and/or a bouncing chamber driving component and/or barrel driving component.

Alternatively, the flange driving component can comprise any at least one other driving component which translates movement along the optical axis in a movement perpendicular to the optical axis.

The barrel driving component can comprise at least one mechanical barrel coupling component, for example a barrel groove couple the ciliary mass to the barrel driving component.

The variable power lens comprises at least one lens component which is a variable power lens component to provide variable optical power of which the degree of power depends on the degree of movement of the mechanical construction in a lateral direction. Such variable power lens component can be a combination of at least two optical elements with each element comprising at least one free-form optical surface with the combination of optical surfaces providing variable optical power of which the degree of power depends on the degree of mutual movement in opposite directions of the optical elements in a lateral direction, or, alternatively, such variable power lens component can be a combination of at least two optical elements with each element comprising at least one largely spherical surface with the combination providing variable optical power of which the degree of optical power depends on the degree of movement, in an axial direction, of the optical elements in opposite directions, or, alternatively, such variable power lens component can be a radially flexible lens component providing variable optical power of which the degree of optical power depends on the degree of movement of the mechanical construction in a lateral direction.

Also, the variable power lens can comprise at least one lens component which is a fixed power lens component providing variable optical power to the eye which power depends on the position of the lens component in a plane perpendicular to the optical axis which fixed power lens component can be a lens providing variable optical power to the eye which power depends on the position of the lens component in a plane along the optical axis, for example, a lens of gradually progressing optical power which power progresses gradually in a direction along the direction of movement of the lens, a progressive lens, or, alternatively, a lens of stepwise progressing optical power which power progresses in steps in a direction along the direction of movement of the lens, a multifocal lens, for example a trifocal lens. Also, such fixed power lens component can be a single largely spherical lens moving along the optical axis.

Any accommodating lens disclosed in the present document the lens can also comprise at least one lens of fixed optical power to provide fixed correction of any other aberration of the eye other than defocus, for example an astigmatism and/or comprise at least one lens of fixed optical power adapted to provide fixed correction of refraction of the aphakic eye. Any of the lens embodiments disclosed in the present document can be designed as a add-on accommodating, piggy back, intraocular lens which provides accommodating power to the eye which eye also contains a refractive lens, for example, the natural lens of the eye or, alternatively, an intraocular lens, for example a monofocal intraocular lens in which cases the refractive lens can be, for example, in the capsular bag of the eye and the accommodating intraocular lens, the add-on lens, positioned at the sulcus plane, in front of the capsular bag.

When multiple optical elements such as lenses are described they may be coupled mutually through an elastic connection, which allows the lenses to move with respect to one another (particularly without forcing the other lens to move as well). Alternatively the elements are not connected mutually at all, such that they can move independently as well.

Furthermore, the accommodating lens can be coupled to at least one intraocular artificial mechanical driving component, for example, a MEMS, a MicroElectroMechanical System, and/or, the variable power lens can comprise any intraocular artificial variable power lens, for example, an optical system including at least one artificial liquid crystal lens with such artificial components and constructions driven, for example, providing leverage of movements of the movements of the eye component, and/or, controlled by movement of at least one eye component via at least one mechanical component as disclosed in the present document.

### DETAILED DESCRIPTION OF THE PREFERRED EMBODIMENTS

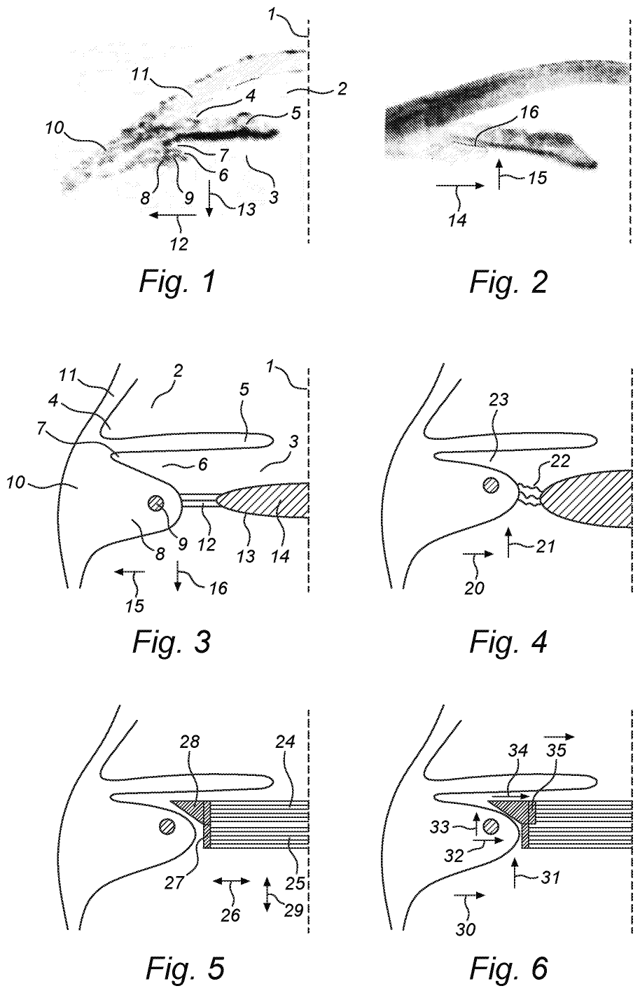

FIG. 1, as in prior art, shows an actual image of the section of the eye showing the ciliary mass and iris, with the optical axis, 1, the anterior chamber, 2, the posterior chamber, 3, the anterior chamber angle, 4, the iris, 5, the sulcus, in this figure, open sulcus, 6, the sulcus root, 7, the ciliary mass, 8, the ciliary muscle fibers, 9, the sclera, 10, which, at the top, merges into the cornea, 11. The zonulae, capsular bag, and natural lens are not visible in this actual image. The muscle is relaxed which causes the ciliary mass to move outward, 12, and backward, 13, which ciliary movements open the sulcus, and, not shown in this image, stretch/pull the zonulae and capsular bag which pull the natural, gel-like, lens in a flat shape which results in a lens of relatively low optical power which provides the eye with a sharp vision at a far distance.

FIG. 2, as in prior art, and with reference to FIG. 1, shows the ciliary muscle contracted, the ciliary mass moved inward, 14, toward the optical axis and forward, 15, towards the iris, which movements close the sulcus, 16, and, not shown in this image, relax the zonulae which allow the natural lens to regain its natural expanded shape which results in a lens of relatively high optical power providing the eye with sharp vision at a nearer distance.

FIG. 3, as in prior art, shows a schematic image of the section of the eye showing the ciliary mass and iris with the optical axis, 1, the anterior chamber of the eye, 2, the posterior chamber of the eye, 3, the anterior angle, 4, the iris, 5, the, in this figure, open, sulcus, 6, the sulcus root, 7, the ciliary mass, 8, the ciliary muscle, 9, the sclera, 10, which, at the top, merges into the cornea, 11, the zonulae, 12, connecting the ciliary mass to the capsular bag, 13, which bag contains the natural lens, 14. The muscle is relaxed, the ciliary mass moved in an outward, 15, and in an backward, 16, position which movements stretch/pull the zonulae, 12, and tighten the capsular bag, 13, which flattens the gel-like natural lens, 14, resulting in a lens of low optical power for sharp vision at a far distance. Note that the ciliary muscle is, in reality, a fragmented collection of single muscular fibers distributed over ciliary mass which distribution is, illustrative purposes, represented in this and following schematics as a single muscle.

FIG. 4, as in prior art, with FIG. 3, shows the muscle contracted, the ciliary mass moved inward, 20, toward the optical axis, and forward, 21, toward the iris, finally narrowing and often even closing the sulcus, which movements relax the zonulae, 22, close the sulcus, 23, which allows the natural lens to regain its natural more rounded shape which results in a lens of relatively high optical power for sharp vision at near distance.

FIG. 5 shows the positioning of the mechanical construction, and variable power lens coupled thereto, of, in this example, an accommodating lens with wedge shaped flange driving component, 28, and a barrel driving component, 27. The variable power lens comprises a variable power lens with two optical elements, 24, 25, which in combination provide variable optical power of which the degree of power depends on the degree of mutual shift, 26, of the elements in a lateral direction. The ciliary muscle is relaxed and exerts low force on the barrel driving component, 27, in a lateral direction and on the flange driving component, 28, the axial direction, 29. The mechanical construction and the variable power lens coupled thereto are expanded outward and the lens provides relatively low optical power to the eye for sharp vision at far distance. The wedge shaped flange driving component, 28, and the barrel driving component, 27 may for instance be coupled mutually by an elastic connection (non-shown).

FIG. 6, with FIG. 5, shows the ciliary muscle contracted and the ciliary mass moved inward, 30, and forward, 31, and exerts force on the barrel driving component in a lateral direction, 32, moving the mechanical construction inward and on the, in this example, wedge shaped flange driving component, 33, in an axial direction which force is translated, by closure of the sulcus and compression of the wedge shaped flange in between the ciliary mass and the iris, into a force perpendicular to the optical axis, 34. The mechanical construction and variable power lens are compressed inwards, the optical elements move mutually, in opposite directions, in a lateral direction, 35, and the lens provides relatively high optical power to the eye for sharp vision at near distance.

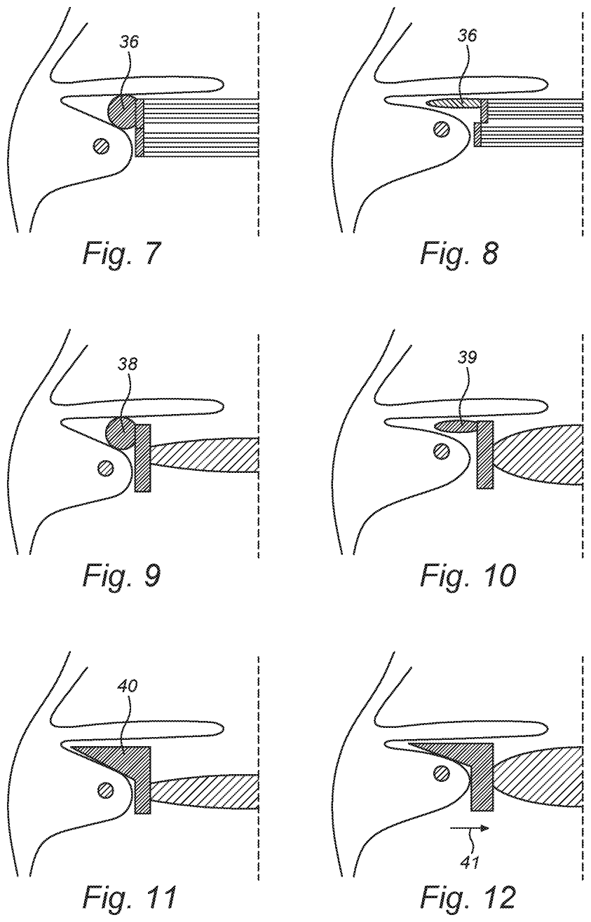

FIG. 7 shows the positioning of the mechanical construction and variable power lens of, in this example, an accommodating lens with a bouncing chamber driving component, 36, and a barrel driving component. In this example the lens comprises a variable power lens with two optical elements. The ciliary muscle is relaxed and exerts a low force on the barrel driving component in a lateral direction and on the bouncing chamber component, the axial direction, allowing expansion of the bouncing chamber component so the mechanical construction and the variable power lens coupled thereto are expanded outward and the lens provides relatively low optical power for sharp vision at far distance.

FIG. 8, with FIG. 7, shows the ciliary contracted and exerting a force, in the axial direction, on the bouncing chamber, and a force, in a lateral direction, on the barrel driving component, the sulcus is closing so the chamber is compressed, 37, which translates the force along the optical axis into a force perpendicular to the optical axis which force compresses the mechanical construction and the variable power lens coupled thereto inwards and the lens provides relatively high optical power to the eye for sharp vision at near distance.

FIG. 9 shows the positioning of the mechanical construction and variable power lens of, in this example, an accommodating lens with an expanded bouncing chamber driving component, 38. In this example the variable power lens comprises a single radially flexible lens component. The ciliary muscle is relaxed and exerts a low force, the axial direction, on the bouncing chamber allowing expansion of the chamber, and a low force on the barrel driving component in a lateral direction, the mechanical construction and variable power lens coupled thereto are expanded outward and the lens provides relatively low optical power to the eye for sharp vision at far distance.

FIG. 10, with FIG. 9, shows the ciliary muscle contracted and exerting a force, the axial direction, on the bouncing chamber compressing the chamber, 39, and a force perpendicular to the optical axis on the barrel driving component, the mechanical construction and variable power lens coupled thereto moved inward and the lens provides relatively high optical power to the eye for sharp vision at near distance.

FIG. 11 shows the positioning of the mechanical construction and variable power lens of, in this example, an accommodating lens with the mechanical construction comprising a wedge shaped flange driving component, 40, and a barrel driving component. In this example the variable power lens comprises a radially flexible lens component. The ciliary muscle is relaxed and exerts a low force, the axial direction, on the wedge shaped flange driving component and on the barrel driving component, the mechanical construction and variable power lens coupled thereto are expanded outward and the lens provides relatively low optical power to the eye for sharp vision at far distance.

FIG. 12, with FIG. 11, shows the ciliary muscle contracted and exerting a high force, the axial direction, on the wedge shaped flange driving component and a high force on the barrel driving component in a lateral direction, the mechanical construction and variable power lens moved inward, 41, compressing the mechanical construction and the variable power lens coupled thereto providing relatively high optical power to the eye for sharp vision at near distance.

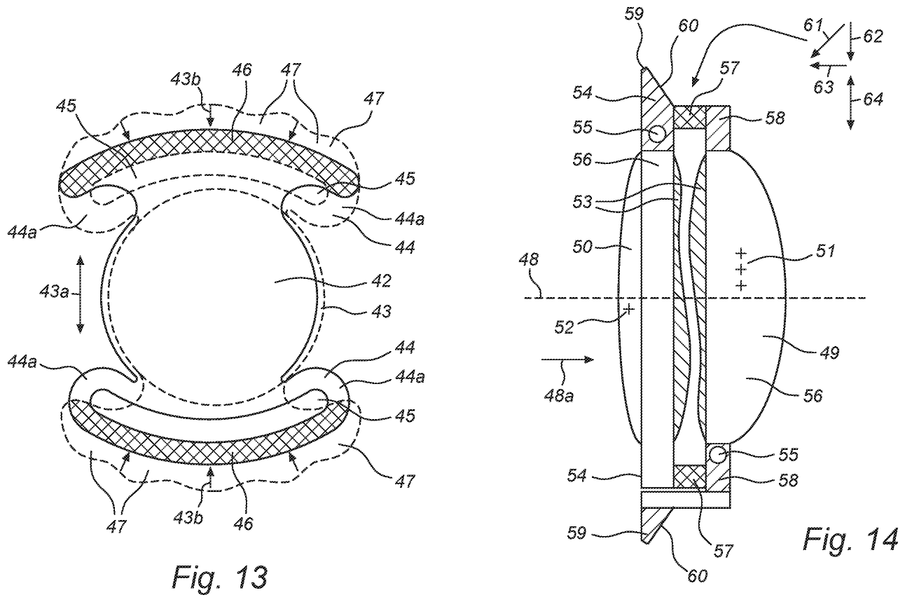

FIG. 13 (variable power lens is prior art) shows a full illustration of an accommodating lens with an variable power lens comprising two optical elements, 42, the anterior optical element and, 43, the posterior element, illustrated in dotted lines, which elements are coupled by a connection, 46, and which elements move mutually, in opposite directions, 43a, in a lateral direction, 43b, driven by the ciliary muscle. The lens comprises, in this example, two mechanical constructions, also: haptics, 44, which each include a hinge, 44a, formed by a curved fenestration, 45, into the body of the mechanical construction. The flange, 47, extends the rim of the anterior, 42, which flange comprises, in this example, waves, 47, on the rim, which waves prevent rotation of the accommodating lens in the sulcus and which flange comprises the mechanical flange driving component (see also explanation related to FIG. 14).

FIG. 14 shows a full illustration of a side view of the lens illustrated in FIG. 13, with the optical axis, 48 and two elements, 56, coupled by a connection, 57. The variable power lens of the lens comprises an anterior lens, 50, of weak optical power lens, 52, and a posterior lens, 49, of high optical power, 51 and a combination of two complementary free form optical surfaces, 53. The mechanical constructions, 54, comprise fenestrations, 55, a mechanical barrel driving component, 58, and a mechanical flange driving component, 54, the transferring component, which, in general, not fully enters the sulcus and with the tapered flange comprising, at, in this example, an anterior surface tip, 59, the tip of the driving component, which, in combination with the rest of the flange driving component, 60, enters the sulcus for driving. The tip, of the driving components is, in this example, chamfered to prevent chafing of the pigment layer with the driving component driven by, in this example, the ciliary muscle, 61, of which the force can be represented by two force vectors, 62, 63, into a force/movement in only a direction perpendicular to the ciliary muscle, 64. In FIG. 14, the bottom tapered flange 59 is connected to the posterior lens, 49, by an unnumbered connection. When the flanges, 59, move towards each other, the posterior lens, 49, moves upwards, and/or the anterior lens, 52, moves downward, wherein the mutual movement causes a change in optical power. The connection, 57, between the two may be an elastic connection that allows this mutual movement. Clearly, in such variable power lens comprising two elements the driving component, on one side of the lens component, is attached to a first optical element, and wherein the driving component, on a second side opposite the one side in a plane perpendicular to the optical axis, is attached to a second, different, optical element.

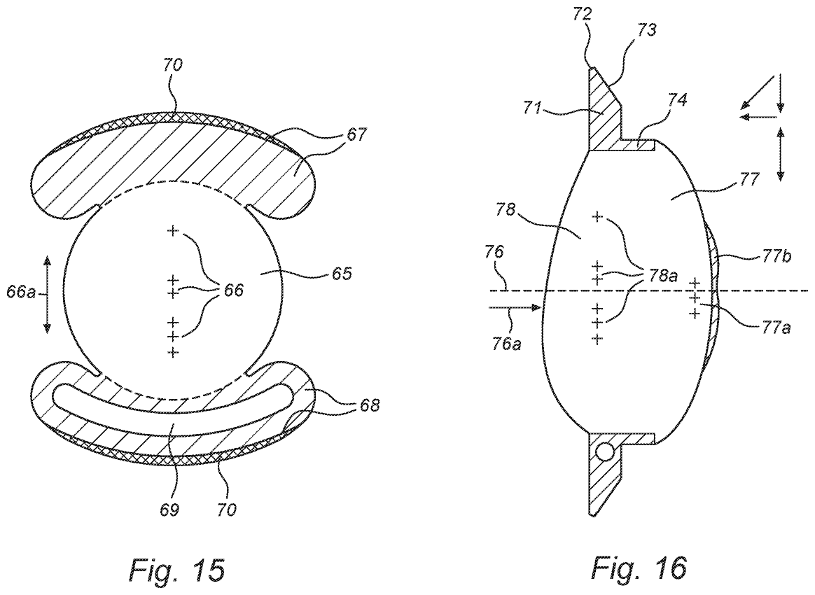

FIG. 15 (variable power lens is prior art), with FIG. 15, shows a full illustration of an accommodating lens with an variable power lens comprising a single optical element, in this example a lens, 65, of gradually increasing optical power, 66, which power progresses in the direction of movement, 66a, of the optical element. One of the mechanical constructions provides flexibility to the variable power lens and comprise a hinge, 68, and fenestration, 69, to allow movement of the lens, the other mechanical construction, 67, lacks such hinge and provides a stiff connection to the variable power lens. The mechanical flange driving component, 70, is further illustrated in FIG. 16.

FIG. 16 shows a full illustration of a side view of the lens illustrated in FIG. 15, with the optical axis, 76, and the direction of the incoming light beam, 76a, two optical surfaces with the anterior surface, 78, providing progressive optical power, 78a, and the posterior optical surface, 77, providing a fixed optical power, 77a, and, in case required, a correcting free-form optical surface, 77b, to correct for optical aberrations due to misalignment of the optical axis of the variable power lens versus the optical axis of the eye which free-form surface can also be include in the anterior optical surface. The mechanical construction, 71, comprises minor chamfering of the front side of the flange, 72, slanting of the backside of the flange, the mechanical flange driving component, 73, and the mechanical barrel driving component, 74. For functioning of the mechanical construction refer to FIG. 15.

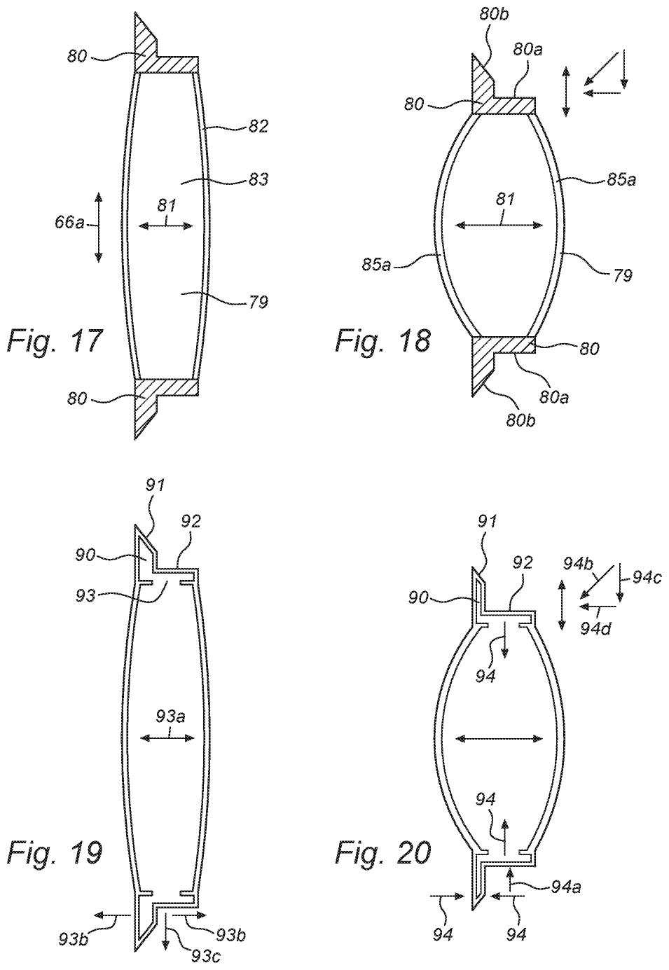

FIG. 17 shows a full illustration of an accommodating lens with an variable power lens comprising a single radially flexible lens component 79. For functioning of the mechanical construction, 80, refer to FIG. 15, except that the mechanical construction does not comprise a hinge/fenestration, comprises two relatively stiff haptics, so that that the variable power lens can be symmetrically driven, meaning: the variable power lens moves equal distances at both sides and the variable power lens remains centered versus the optical axis of the eye. In this example the variable power lens, 83, comprises a radially flexible lens, 79, gradually increasing optical power when radially compressed by the mechanical construction, for example a gel or an oil or any other significantly flexible substance contained in a capsule, 82. In this figure the variable power lens is relaxed, the lens flattened, 81, and providing the eye with relatively low optical power for sharp vision at far distance.

FIG. 18, with FIG. 17, shows the accommodating lens with an variable power lens comprising a single radially flexible lens component 79. For functioning of the mechanical construction, 80, refer to FIG. 15. In this figure the variable power lens is contracted, via the mechanical construction, 80, by the force of the ciliary muscle, not shown, on the mechanical barrel driving component, 80a, and on the mechanical flange driving mechanism, 80b, the anterior and posterior radii, 85a, increased, rounded, providing the eye with relatively high optical power for sharp vision at near distance. For the directional vectors refer to FIG. 14.

FIG. 19, see also FIGS. 17-18, shows the accommodating lens with an variable power lens, a single radially flexible lens component, in the relaxed, flattened shape to provide relatively low optical power to provide the eye with sharp vision at far distance, driven by a mechanical construction which comprises a combination of a wedge shaped flange driving component, 91, and a bouncing chamber driving component, 90, and a barrel driving component, 92, which component is connected, via a channel, 93, to the radially flexible lens component. With the mechanical construction and the variable power lens in a relaxed state the bouncing chamber is expanded in both the axial, 93b, and in a lateral direction, 93c.

FIG. 20, with FIG. 19, shows the accommodating lens with an variable power lens, a single radially flexible lens component, in the compressed, rounded, shape to provide relatively high optical power to provide the eye with sharp vision at near distance, driven by a mechanical construction compressed by a force at an angle, 94b, in between perpendicular and along the optical axis, which force both (a) compresses, by force along the optical axis, 94d, the bouncing chamber causing the any relatively liquid substance in the chamber and lens capsule to largely flow into the variable power lens, 94, resulting in inflation/rounding of the lens component and thus increasing the optical power of the radially flexible lens component (b) pushes the flange driving component in an axial direction, 94d, resulting in compression of the lens component in a lateral direction, further increasing optical power of the radially flexible lens component and, (c) pushes the barrel driving component, 92, in a lateral direction resulting in compression of the lens component in a lateral direction, even further increasing optical power of the radially flexible lens component.

So, the present invention discloses an accommodating intraocular lens, providing accommodation to an eye to an eye with the eye and the lens having the same optical axis, with the accommodating lens comprising: at least one variable power lens comprising at least one variable power lens to provide optical modification of an incoming light beam which lens component is coupled to at least one mechanical construction to transfer movement of an eye driving component in the eye to movement of at least one lens component of the variable power lens with the mechanical construction comprises at least one driving component to provide translation of movement of the driving component in an axial direction into movement of the mechanical construction in a lateral direction and with the mechanical construction comprising at least one transferring component to provide transfer of the movement in lateral direction of the driving component to the variable power lens. The driving component and the transferring component can be combined in a single component but, alternatively, can be separate components.

The driving component can be a flange driving component for providing said translation of movement, wherein, for instance, the flange driving component comprises a flange arranged to be positioned at least partially in the sulcus of the eye. The part of the flange driving component is, in this example, a transferring component, with the wedge shaped flange driving component for providing said translation of movement preferably the wedge shape flange driving component which tapers towards its free end. The eye driving component can be a natural component of the eye, for example, the ciliary mass of the eye, or, alternatively, can be an artificial driving component, for example an intraocular micro-electro-mechanical system.

The driving component can be at least one bouncing chamber driving component for providing said translation of movement which preferably is arranged to deform upon movement of the ciliary mass, and/or the zonular system, and transfers movement to the variable power lens. The mechanical construction can also comprise at least one barrel driving component for providing transfer of movement of the ciliary mass in a lateral direction to movement of the mechanical component in a lateral direction, wherein the barrel driving component, for instance, comprises at least one mechanical barrel coupling component, for example a barrel groove, to couple the ciliary mass to the barrel driving component, preferably with the barrel driving component is coupled at least partially to at least one of the lens components by a transferring component. The mechanical construction can comprise any combination of a wedge shaped flange driving component and a bouncing chamber driving component and a barrel driving component.

The variable power lens component can be a variety of variable power lenses, for example a single lens of fixed optical power comprising at least one largely spherical surface which provides variable optical power to the eye of which the degree of optical power depends on the degree of movement, in an axial direction, of the lens along the optical axis, or, alternatively, can be a combination of at least two optical elements with each element comprising at least one largely spherical surface wherein the combination provides variable optical power of which the degree of optical power depends on the degree of movement, in an axial direction, along the optical axis, of the optical elements in opposite directions, or, alternatively, can a combination of at least two optical elements with each element comprising at least one free-form optical surface, wherein the combination of optical surfaces provides variable optical power of which the degree of power depends on the degree of mutual movement in opposite directions of the optical elements in a lateral direction, or, alternatively, can be the variable power lens component can comprise a radially flexible lens comprising two optical surfaces with the lens providing variable optical power of which the degree of optical power depends on the degree of movement of the mechanical construction in a lateral direction which movement provides a change in the radius of at least one optical surface of the lens.

When the variable power lens component is a combination of at least two optical elements, the driving component, on one side of the lens component, maybe attached to a first optical element. The driving component, on a second side opposite the one side in a plane perpendicular to the optical axis, may be attached to a second, different, optical element. The driving component in the eye can be a natural driving component wherein the degree of movement of the variable power lens depends on the degree of movement of at least one eye component, for example the driving component can be the ciliary mass of the eye or the zonular system of the eye, or, alternatively, the driving component can be an artificial driving component wherein the degree of movement of the variable power lens depends on the degree of movement of at least one component of the artificial driving component, for example any micro-electro-mechanical system (also: ‘MEMS’) with the artificial mechanical driving component which can be configured to move at least one variable power lens comprising at least one intraocular artificial variable power lens, such as a variable power lens including at least one artificial liquid crystal lens.

Finally, the lens can also comprises at least one posterior anchoring component which component provides anchoring of the lens by coupling to the rim of the capsullorhexis.

1. Accommodating intraocular lens, for providing accommodation to an eye with the eye and the lens having the same optical axis, with the accommodating lens comprising: at least one variable power lens comprising at least one variable power lens to provide optical modification of an incoming light beam which lens component is coupled to at least one mechanical construction to transfer movement of an eye driving component in the eye to movement of at least one lens component of the variable power lens characterized in that the mechanical construction comprises at least one driving component to provide translation of movement of the driving component in an axial direction into movement of the mechanical construction in a lateral direction and with the mechanical construction comprising at least one transferring component to provide transfer of the movement in lateral direction of the driving component to the variable power lens.

2. Accommodating lens according to claim 1 characterized in that the driving component and the transferring component are the same component which component provides translation of movement as well as transfer of movement.

3. Accommodating lens according to claim characterized in that the driving component is a flange driving component for providing said translation of movement, wherein, for instance, the flange driving component comprises a flange arranged to be positioned at least partially in the sulcus of the eye.

4. Accommodating lens according to claim 3 characterized in that the driving component is a wedge shaped flange driving component for providing said translation of movement, wherein, preferably, the wedge shape flange driving component tapers towards its free end.

5. Accommodating lens according to claim 1 characterized in that the driving component is at least one bouncing chamber driving component for providing said translation of movement wherein, preferably, the bouncing chamber driving component is arranged to deform upon movement of the ciliary mass and transfers movement to the variable power lens.

6. Accommodating lens according to claim 1, characterized in that the mechanical construction also comprises at least one barrel driving component for providing transfer of movement of the ciliary mass in a lateral direction to movement of the mechanical component in a lateral direction, wherein the barrel driving component for instance comprises at least one mechanical barrel coupling component, for example a barrel groove, to couple the ciliary mass to the barrel driving component, wherein preferably the barrel driving component is coupled at least partially to at least one of the lens components.

7. Accommodating lens according to claim 1, characterized in that the mechanical construction comprises any combination of a wedge shaped flange driving component and a bouncing chamber driving component and a barrel driving component.

8. Accommodating lens according to claim 1, characterized in that
the variable power lens component is a single lens of fixed optical power comprising at least one largely spherical surface which provides variable optical power to the eye of which the degree of optical power depends on the degree of movement, in an axial direction, of the lens along the optical axis.

9. Accommodating lens according to claim characterized in that
the variable power lens component is a combination of at least two optical elements with each element comprising at least one largely spherical surface wherein the combination provides variable optical power of which the degree of optical power depends on the degree of movement, in an axial direction, along the optical axis, of the optical elements in opposite directions.

10. Accommodating lens according to any combination of claim 1 characterized in that the variable power lens component is a combination of at least two optical elements with each element comprising at least one free-form optical surface, wherein the combination of optical surfaces provides variable optical power of which the degree of power depends on the degree of mutual movement in opposite directions of the optical elements in a lateral direction.

11. Accommodating lens according to claim 1, characterized in that the variable power lens component is a combination of at least two optical elements, wherein the driving component, on one side of the lens component, is attached to a first optical element, and wherein the driving component, on a second side opposite the one side in a plane perpendicular to the optical axis, is attached to a second, different, optical element.

12. Accommodating lens according to claim 1 characterized in that the variable power lens component comprises a radially flexible lens comprising two optical surfaces with the lens providing variable optical power of which the degree of optical power depends on the degree of movement of the mechanical construction in a lateral direction which movement provides a change in the radius of at least one optical surface of the lens.

13. Accommodating lens according to claim 1 characterized in that the eye driving component is a natural driving component wherein the degree of movement of the variable power lens depends on the degree of movement of at least one eye component.

14. Accommodating lens according to claim 13 characterized in the the eye driving component is the ciliary mass of the eye.

15. Accommodating lens according to claim 1 characterized in that the eye driving component is an artificial driving component wherein the degree of movement of the variable power lens depends on the degree of movement of at least one component of the artificial driving component.

16. Accommodating lens according to claim 15 characterized in that the mechanical driving component is a micro-electro-mechanical system.

17. Accommodating lens according to claim 16 characterized in that the artificial mechanical driving component is configured to move at least one variable power lens comprising at least one intraocular artificial variable power lens, such as a variable power lens including at least one artificial liquid crystal lens.

18. Accommodating lens according to claim 1 characterized in that the lens also comprises at least one posterior anchoring component which component provides anchoring of the lens by coupling to the rim of the capsullorhexis.
